(19) United States
(12) Patent Application Publication
(10) Pub. No.: US 2019/0125577 A1
(43) Pub. Date: May 2, 2019

(54) APPARATUS FOR CONTROLLING BODY TEMPERATURE USING THERMOELECTRIC ELEMENT

(71) Applicant: Ryungil CHOI, Goyang-si (KR)

(72) Inventor: Ryungil CHOI, Goyang-si (KR)

(21) Appl. No.: 16/096,065

(22) PCT Filed: May 31, 2016
(86) PCT No.: PCT/KR2016/005743

§ 371 Date: Oct. 24, 2018

(30) Foreign Application Priority Data
May 30, 2016 (KR) 10-2016-0066246

(51) Int. Cl. A61F 7/00

(52) U.S. Cl. CPC A61F 7/007; A61F 7/0085; A61F 2007/0086; A61F 2007/0055; A61F 2007/0057; A61F 2007/0076

(57) ABSTRACT

The present invention relates to an apparatus for controlling body temperature using a thermoelectric element, the apparatus including: a chamber unit including a water container configured to store water; a temperature control unit configured to cool or heat water; a circulation unit including a water circulation line configured such that the cooled or heated water discharged from the temperature control unit is circulated therethrough to flow back to the temperature control unit; a pad unit configured to contain a part of the water circulation line and to increase or decrease the body temperature of a human; and a control unit configured to control the operation of the temperature control unit; wherein the temperature control unit includes a cartridge part and a body part, and the body part includes a thermoelectric unit including a thermoelectric element and a heat sink plate.

### TECHNICAL FIELD

The present invention relates to an apparatus for controlling body temperature using a thermoelectric element.

### BACKGROUND ART

All patients require the management of body temperature. In particular, an operating room, an intensive care unit, and a recovery room need to be each equipped with an apparatus for controlling body temperature that has both heating and cooling functions. In practice, one degree of patient body temperature is important enough to affect a patient's life.

Meanwhile, technology related to an apparatus for controlling body temperature has been disclosed in International Patent Publication No. 2007-089293. However, in the disclosed apparatus for controlling body temperature, to which a body temperature control system has been applied, a water container and pipelines through which water flows are fixedly integrated with the apparatus, and thus tap water cannot be used due to a failure attributable to sediments, etc. but only distilled water must be used. In this case, a large amount of distilled water corresponding to about 12 liters of water must always be provided, and a user requires a lot of power when he or she pours water into the apparatus. Furthermore, the apparatus adopts a method using a refrigerant for heating and cooling, and thus the use of the apparatus is limited in terms of environmental protection and regulations. Moreover, a lot of noise is generated due to a motor required for circulation, and thus it is difficult to use the apparatus in various departments of a hospital that require the stability of patients.

### DISCLOSURE

### Technical Problem

One embodiment provides an apparatus for controlling body temperature using a thermoelectric element, which is environmentally friendly and almost free from noise and the risk of fire, thereby providing a safe and comfortable environment to a patient and a user, which can control body temperature within a range from a lower temperature to a higher temperature, can considerably reduce the time required for cooling and heating, and enables tab water to be used, thereby improving the convenience of a user, which can accurately transfer a set temperature to a user and enables the user to accurately recognize the set temperature in the maintenance of body temperature, thereby improving the efficiency of the control of body temperature, and which provides the control of body temperature and can prevent a bedsore with excellent performance.

### Technical Solution

According to an embodiment, there is provided an apparatus for controlling body temperature using a thermoelectric element, the apparatus including: a chamber unit including a water container configured to store water; a temperature control unit configured to cool or heat water supplied from the chamber unit; a circulation unit including a water circulation line configured such that the cooled or heated water discharged from the temperature control unit is circulated therethrough to flow back to the temperature control unit; a pad unit configured to contain a part of the water circulation line and to increase or decrease the body temperature of a human who comes into contact therewith; and a control unit configured to control the operation of the temperature control unit; wherein the temperature control unit includes a cartridge part and a body part located on the top surface of the cartridge part, and the body part includes a thermoelectric unit including a thermoelectric element located on the top surface of the cartridge part and a heat sink plate located on the top surface of the thermoelectric element; and wherein the pad unit through which the cooled or heated water is circulated has an adjusted temperature ranging from 4 to 50° C., and the difference between a maximum temperature and a minimum temperature within an adjusted temperature range of the pad unit ranges from 41 to 46° C.

The time required to cool water from 25° C. to 8° C. via the temperature control unit may range from 10 to 30 minutes.

The chamber unit may further include a sensor located independently of the water container, an indicator mounted on a side surface of the water container, or a combination thereof.

The body part may further include a cover plate located on at least one of the faceable surfaces of the thermoelectric unit; and the cover plate may be located on at least the top surface of the thermoelectric unit, and the cover plate located on the top surface of the thermoelectric unit may include a fan.

The ratio between sectional areas based on facing surfaces of the cover plate located on the top surface of the thermoelectric unit and the thermoelectric unit may range from 1:1 to 1.5:1, and the cover plate located on the top surface of the thermoelectric unit may include one to four fans.

The cover plate may be located on the top surface, left side surface, and right side surface of the thermoelectric unit.

The cover plate may be located at a distance from the faceable surfaces including the top surface of the thermoelectric unit.

The cover plate further located on at least one of the front surface, back surface, left side surface, and right side surface of the thermoelectric unit may include at least one fan, at least one hole, or a combination thereof, and the cover plate including the hole further may include a roof above the hole.

The cartridge part may have a structure that is detached from the body part.

The cartridge part may include: a flow passage body including an inlet configured to receive water, a flow passage region configured such that a flow passage configured to cool or heat water is formed, and an outlet configured to discharge the cooled or heated water; and temperature sensors located below the bottom surface of the flow passage body.

The temperature sensors may include: a first temperature sensor located on an outlet side below the bottom surface of the flow passage body, and configured to measure the temperature of the cooled or heated water; and a second temperature sensor located on an inlet side below the bottom surface of the flow passage body, and configured to measure the temperature of water that flows into the temperature control unit after being circulated through the pad unit.

The ratio between sectional areas based on facing surfaces of the flow passage body and the thermoelectric unit may range from 1:1 to 1:1.5.

The part of the water circulation line contained in the pad unit may have a zigzag structure, a honeycomb structure, or a combination structure thereof.

The apparatus for controlling body temperature may further include an air unit including an airline; the pad unit further may contain a part of the airline independently of the water circulation line; and the control unit may further control the operation of the air unit.

The air unit may further include an air manipulation unit configured to enable entry and discharge of air to be repeatedly performed.

The part of the airline contained in the pad unit may have a fork structure in a direction parallel to a direction in which air is injected.

### Advantageous Effects

The apparatus for controlling body temperature using a thermoelectric element is environmentally friendly and almost free from noise and the risk of fire because it does not employ a refrigerant or hot wire, and thus a safe and comfortable environment can be provided to a patient and a user. Furthermore, the apparatus for controlling body temperature using a thermoelectric element can control body temperature within a range from a lower temperature to a higher temperature and can considerably reduce the time required for cooling and heating, and thus it can be used in anywhere requiring medical measures, including various departments of a hospital requiring the maintenance of body temperature and therapeutic hypothermia. Furthermore, tab water can be used, and thus the convenience of a user can be considerably improved. Furthermore, a set temperature can be accurately transferred to and recognized by a user in the maintenance of body temperature, and thus the accurate and rapid control of body temperature can be performed. Moreover, body temperature can be controlled, and the bedsore of a patient can be prevented with excellent performance.

### DESCRIPTION OF DRAWINGS

FIG. 1 is a schematic diagram showing the configuration of an apparatus for controlling body temperature using a thermoelectric element according to one embodiment;

FIG. 2 is a schematic diagram schematically showing a temperature control unit according to one embodiment;

FIG. 3 is a sectional view of a region taken along line A-A′ of FIG. 2; and

FIG. 4 is a schematic diagram showing the configuration of an apparatus for controlling body temperature using a thermoelectric element according to another embodiment.

### BEST MODE

Embodiments will be described in detail below so that those having ordinary knowledge in the art can easily practice the present invention. However, embodiments may be implemented as various different forms, and are not limited to the embodiment described herein.

In the drawings, thicknesses will be enlarged in order to clearly indicate various layers and regions. Throughout the specification, the same reference symbols are assigned to similar parts. When a part, such as a layer, a film, a region, a plate, or the like, is described as being “on” or “above” another region, this includes not only a case where the former part is “directly on” or “directly above” the other part but also a case where a third part is disposed therebetween. In contrast, when a part is “directly above” another part, this means that a third part is not present therebetween. Furthermore, when a part, such as a top surface, a bottom surface, a front surface, a back surface, a left side surface, a right side surface, or the like, is described as being “under” or “beneath” another region, this includes not only a case where the former part is “directly under” or “directly beneath” the other part but also a case where a third part is disposed therebetween.

Furthermore, based on a front surface, a surface above the front surface is defined as a top surface, a surface below the front surface is defined as a bottom surface, a surface opposite to the front surface is defined as a back surface, a surface on the left of the front surface is defined as a left side surface, and a surface on the right of the front surface is defined as a right side surface.

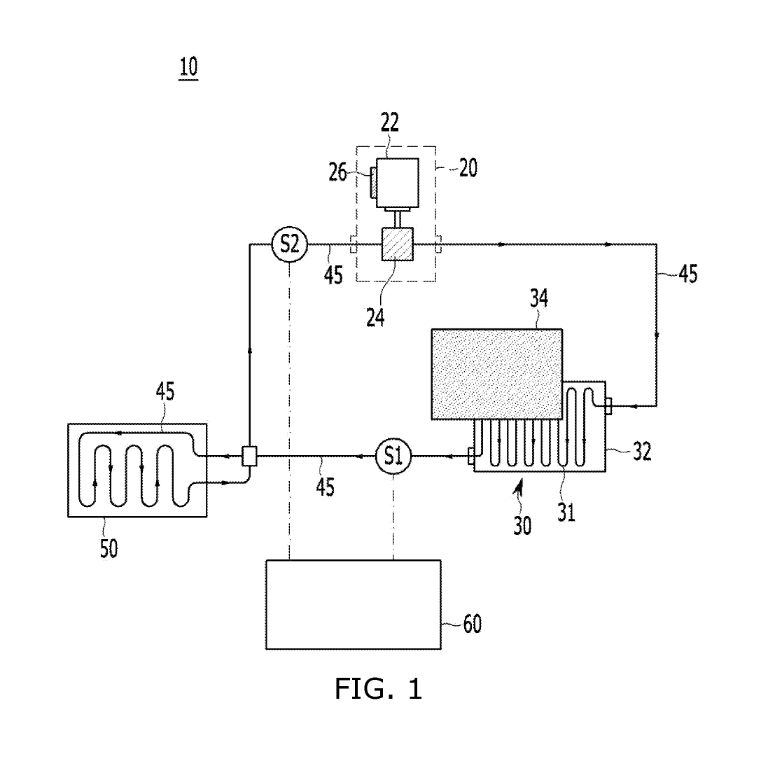

An apparatus for controlling body temperature according to one embodiment will be described with reference to FIG. 1. The drawing to which reference will be made corresponds merely to one form of the apparatus for controlling body temperature according to one embodiment, and the present invention is not limited thereto.

FIG. 1 is a schematic diagram showing the configuration of the apparatus for controlling body temperature using a thermoelectric element according to one embodiment.

Referring to FIG. 1, the apparatus 10 for controlling body temperature according to one embodiment may include a chamber unit 20 configured to store water, a temperature control unit 30 including a thermoelectric element (not shown) configured to cool or heat water supplied from the chamber unit 20, a circulation unit (not shown) configured such that the cooled or heated water discharged from the temperature control unit 30 is circulated therethrough to flow back into the temperature control unit 30, a pad unit 50 configured to contain a part of the circulation unit and to increase or decrease the body temperature of a human who comes into contact therewith, and a control unit 60 configured to control the operation of the temperature control unit 30.

In other words, the process in which water is circulated may be repeated via the apparatus 10 for controlling body temperature according to the embodiment in such a manner that water supplied from the chamber unit 20 is cooled or heated to a set temperature by the thermoelectric element in the temperature control unit 30, the cooled or heated water is circulated through the pad unit 50 via the circulation unit, and then the cooled or heated water flows back into the temperature control unit 30. Via the above-described circulation structure, the body temperature of a human who comes into contact with the pad unit can be maintained at a normal level, and body temperature can be controlled by decreasing or increasing the body temperature. In particular, according to one embodiment, a wider range from a minimum temperature to a maximum temperature in which body temperature can be controlled can be secured by applying the thermoelectric element to the apparatus for controlling body temperature. Accordingly, the present invention is useful for patients requiring the control of body temperature as in the case of therapeutic hypothermia, and the present invention can be usefully used for people requiring medical measures, such as emergency patients, by considerably reducing the time required for cooling or heating.

The temperature control unit 30 including the thermoelectric element will be described in detail later.

The chamber unit 20 may include a water container 22 configured to store water. The water container 22 may be configured in a detachable form so that the water container 22 can be easily replaced. When the water container 22 is configured in a detachable form, water can be directly stored and replenished and tab water can be used, thereby improving the convenience of a user.

According to one embodiment, the cartridge part 32 of the temperature control unit 30 to be described later as well as the water container 22 may be configured in detachable forms that can be detached. A passage configured such that water flows therethrough is formed through the cartridge part 32, which will be described in detail below. In one embodiment, the water container 22 and the cartridge part 32 are configured in detachable forms, and thus tab water can be used. In the conventional apparatus for controlling body temperature, the water container and the water pipelines are fastened in complicated structures, and thus it is difficult to replace them, with the result that only distilled water can be used. The reason for this is that, when the water container and the water pipelines are not replaced for a long time, tab water may cause a problem in the function of the apparatus for controlling body temperature and may stop the operation of the apparatus by blocking a passage due to the settlement of impurities, the coagulation of mineral components, or the like, unlike distilled water. In this case, damage may be imposed on a patient, and there may occur an inefficient situation in which the overall part needs to be repaired and an inconvenient situation in which a user cannot use the apparatus during a repair period. According to one embodiment, it is possible to periodically replace the water container 22 and the cartridge part 32, and thus tab water can be conveniently used without the erroneous operation of the apparatus. Furthermore, the apparatus can be used with a small amount of water, and thus users can conveniently use the apparatus.

The water container 22 may have a size sufficient to store an amount of water larger than the amount of water that flows into and is circulated through the pad unit 50.

The chamber unit 20 may further include a sensor 24, an indicator 26, or a combination thereof in addition to the water container 22. The sensor 24 and the indicator 26 are means for detecting the amount of water, and may be selectively employed to secure a sufficient amount of water required for circulation through the pad unit 50. When the supply of water is insufficient, the erroneous operation of the apparatus occurs. Accordingly, the apparatus may be considered to be safer than a method using a common check valve, and thus the apparatus for controlling body temperature according to one embodiment can be more usefully used as a medical apparatus.

The sensor 24 is a sensor capable of detecting the amount of water, and may be formed at a location independent of the water container 22. In a specific one form, the sensor 24 may detect the amount of water as one of three states including a sufficient water state, a water replenishment requiring state, and a water shortage state, and may transfer the detected amount of water to a user via one of three-state alarms. In the case of the water shortage state, an alarm warning sound may be generated, and there may be provided notification that the apparatus cannot operate. In the case of the water replenishment requiring state, there may be provided notification that the apparatus can operate normally and may enter the water shortage state when it will be continuously used. Accordingly, water is allowed to be replenished in advance, and thus a cause for the erroneous operation of the apparatus or the stoppage of the operation of the apparatus can be prevented in advance.

The indicator 26 is an electronic indicator configured to enable a user to view the state of the amount of water via a variation in color, and may be mounted at a location on a side surface of the water container 22 which can be viewed by the user.

It may be possible to employ both the sensor 24 and the indicator 26 within the chamber unit 20.

The chamber unit 20 may have inlets (not shown) configured to receive water and an outlet (not shown) configured to discharge water, in which case the inlets may include a first inlet (not shown) configured to receive water stored in water container 22 and a second inlet (not shown) configured such that cooled or heated water enters back into the chamber unit 20 for the purpose of circulation therethrough after passing through the pad unit 50.

The chamber unit 20 may be formed in a structure that is separable from the water circulation line 45 of the circulation unit (not shown) to be described later. Furthermore, a check valve (not shown) may be formed in the inlet of the chamber unit 20, more specifically the second inlet, and the outlet. When the chamber unit 20 and the water circulation line 45 are separated from each other, water is prevented from leaking by means of the check valve. The second inlet and outlet of the chamber unit 20 may be connected to each other with a single check valve having male and female connectors interposed therebetween. This check valve is apparent to those skilled in the art, and a description thereof is omitted here. The structure and mounting method of the check valve may be appropriately selected and employed by those skilled in the art.

The temperature control unit 30 is a unit configured to cool or heat water supplied from the chamber unit 20, and may include the cartridge part 32 and a body part 34.

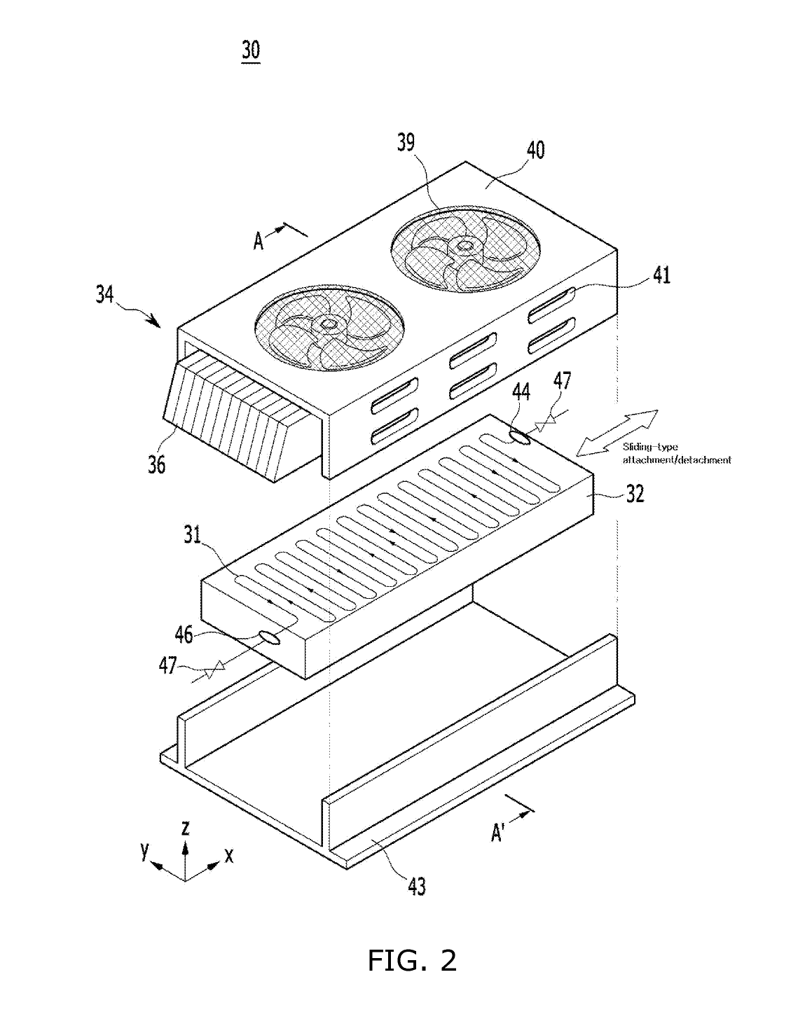

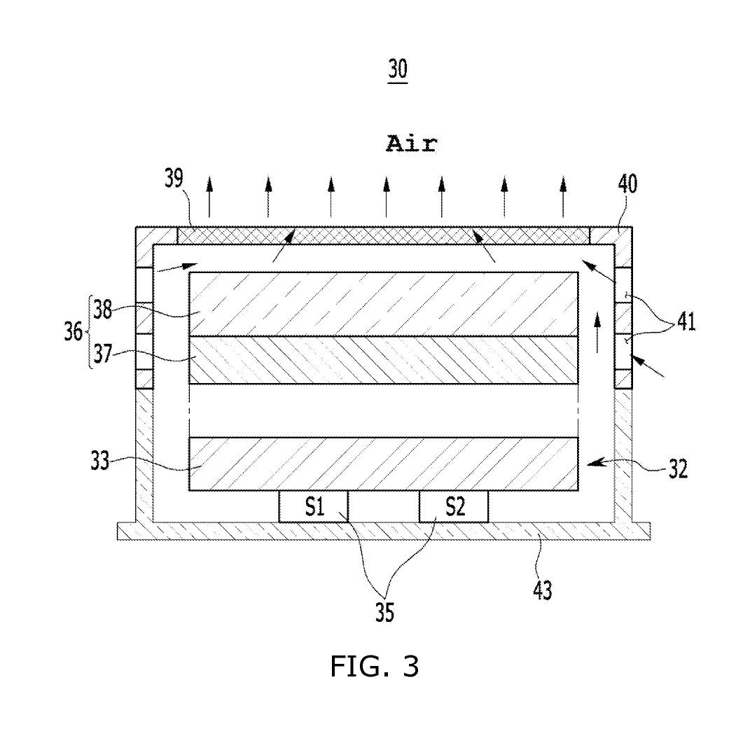

More specifically, the temperature control unit 30 will be described with reference to FIGS. 2 and 3. The drawings to which reference will be made correspond merely to one form of the temperature control unit according to one embodiment, and the present invention is not limited thereto.

FIG. 2 is a schematic diagram schematically showing a temperature control unit according to one embodiment, and FIG. 3 is a sectional view of a region taken along line A-A′ of FIG. 2.

In FIG. 2, x, y and z are shown to refer to the directions of the faceable surfaces of the thermoelectric unit 36. For example, an x direction is a direction referring to the back surface of the thermoelectric unit 36, the direction opposite to the x direction is a direction referring to the front surface of the thermoelectric unit 36, a y direction is a direction referring to the left side surface of the thermoelectric unit 36, the direction opposite to the y direction is a direction referring to the right side surface of the thermoelectric unit 36, a z direction is a direction referring to the top surface of the thermoelectric unit 36, and the direction opposite to the z thermoelectric unit 36 is a direction referring to the bottom surface of the thermoelectric unit 36.

Referring to FIGS. 2 and 3, the temperature control unit 30 according to one embodiment may include the cartridge part 32 and the body part 34 located on the top surface of the cartridge part 32.

A passage configured such that water flows therethrough, i.e., a flow passage, is formed inside the cartridge part 32. Water supplied from the chamber unit 20 passes through the flow passage. The cartridge part 32 may have a structure that is separated from the body part 34. For example, the cartridge part 32 may have a detachable sliding-type structure, but is not limited thereto. When the cartridge part 32 has a sliding-type structure and is detached, the direction in which the cartridge part 32 is detached may be, for example, a direction referring to the front surface of the thermoelectric unit 36 or a direction referring to the back surface, as shown in FIG. 2. The direction is not limited thereto.

As described above, the cartridge part 32 is formed in a detachable form, and, thus, can be periodically replaced. Tab water can be conveniently used without the risk of the erroneously operation or stoppage of the operation of the apparatus.

The body part 34 may include the thermoelectric unit 36.

The thermoelectric unit 36 may include a thermoelectric element 37 located on the top surface of the cartridge part 32 and a heat sink plate 38 located on the top surface of the thermoelectric element 37.

Although the cartridge part 32 is shown as being spaced apart from the bottom surface of the thermoelectric unit 36 by a distance in FIGS. 2 and 3, this merely indicates that the cartridge part 32 can be separated from the body part 34 including the thermoelectric unit 36. When the cartridge part 32 is mounted in the apparatus, the cartridge part 32 and the thermoelectric unit 36 may come into maximum contact with each other for the purpose of efficient thermal conduction.

The thermoelectric element 37 is an element configured to cool or heat water passing through the flow passage within the cartridge part 32 located below the thermoelectric element 37. More specifically, a Peltier element using the Peltier effect may be used as the thermoelectric element 37. The Peltier effect is based on the principle in which, when different metals are attached to each other and power is applied to the attached metals, a heat radiation action occurs on one side and a heat absorption action occurs on the other side according to the polarity of the power.

The apparatus for controlling body temperature according to one embodiment uses the thermoelectric element 37, such as a Peltier element, without using a refrigerant or hot wire. Today, the use of refrigerant gas is limited for the purpose of the protection of an environment due to the depletion of the ozone layer, and serious noise is generated due to the mounting of a motor required for circulation. Furthermore, when a problem occurs in a refrigerant system, the refrigerant system needs to be charged with a refrigerant via a separate company that deals in refrigerant gas, and thus it is inefficient in terms of time and cost and the function cannot be guaranteed after the service. Furthermore, the hot wire used for heating causes high power consumption, has the risk of fire, and may cause a lukewarm burn to a patient. In one embodiment, the thermoelectric element replaces a refrigerant or hot wire, and the present invention can be used for various departments of a hospital, including an intensive care unit where the stability of a patient is essential because the thermoelectric element is environmentally friendly, rarely generates noise, and does not cause the risk of fire. Furthermore, a wider temperature range within which body temperature can be controlled can be secured and cooling and heating can be rapidly performed, and thus the present invention can be usefully used in medical fields requiring both the maintenance of normal body temperature and therapeutic hypothermia.

The heat sink plate 38 may be located on the top surface of the thermoelectric element 37, and may radiate heat generated in the thermoelectric element 37.

The body part 34 may further include a cover plate 40 located on at least one of the faceable surfaces of the thermoelectric unit 36. The faceable surfaces of the thermoelectric unit 36 may include, for example, the top surface, front surface, back surface, left side surface, and right side surface of the thermoelectric unit 36. The cover plate 40 may be located on a single surface of the thermoelectric unit 36, or may be located on a plurality of surfaces thereof.

In this case, when the cover plate 40 is located on two or more adjacent surfaces of the thermoelectric unit 36, the cover plate 40 may be configured in an integrated form in a perpendicular structure. However, the integrated form is not limited to a particular form.

The cover plate 40 may be formed in a flat plate (not shown) structure. One or more fans 39 configured to receive or discharge external air may be mounted on the cover plate 40. Alternatively, one or more holes 41 configured to secure a passage through which external air enters into the plate and to guide air through a direction are formed in the plate. Alternatively, the cover plate 40 may have both the fans 39 and the holes 41. For example, each of the parts of the cover plate 40 corresponding to surfaces of the thermoelectric unit 36 may have any one of the fans 39 and the holes 41.

FIGS. 2 and 3 merely illustrate an example of one form of the temperature control unit, and thus the reference symbols shown in FIGS. 2 and 3 are not limited to the parts shown. Parts having the same terms may be designated by the same reference symbols. Accordingly, in the following, although the fans 39 are shown as being present only in the top surface of the thermoelectric unit 36 in FIGS. 2 and 3, the same reference symbol is used for a fan 39 that is present in a surface that is not shown and that may correspond to the thermoelectric unit 36.

The body part 34 may further include the cover plate 40, and thus a system that can efficiently secure a passage configured to receive and discharge external air may be formed. As a result, the performance of the thermoelectric unit 36 is considerably improved, and thus a temperature range within which body temperature can be controlled can be increased by performing cooling to a lower temperature and heating to a higher temperature and the time required for cooling or heating can be considerably reduced.

More specifically, the cover plate 40 may be located on at least the top surface of the thermoelectric unit 36 among the faceable surfaces of the thermoelectric unit 36. In this case, the cover plate 40 located on the top surface of the thermoelectric unit 36 may include the fans 39. In this case, the cover plate 40 located on the top surface of the thermoelectric unit 36 may be integrated with the fans 39. Heat can be rapidly radiated in such a manner that air entering from surroundings around the thermoelectric unit 36 exits through the fans 39 located in the top surface of the thermoelectric unit 36. Furthermore, air is discharged through the top surface of the thermoelectric unit 36 by the operation of the fans 39 and simultaneously external air around the thermoelectric unit 36 can be easily drawn, and thus an efficient air passage can be secured. Accordingly, the cooling or heating performance of the thermoelectric unit 36, more specifically the thermoelectric element 37, can be considerably improved.

The ratio between the sectional areas of the facing surfaces of the cover plate 40 located on the top surface of the thermoelectric unit 36 and the thermoelectric unit 36, i.e., the ratio between the sectional areas of the facing surfaces of the cover plate 40 and the thermoelectric unit 36, may range from 1:1 to 1.5:1, for example, from 1:1 to 1.4:1, from 1:1 to 1.3:1, from 1:1 to 1.2:1, or from 1:1 to 1.1:1, but is not limited thereto. When the ratio between the sizes of the cover plate on the top surface and the thermoelectric unit falls within the range, a passage system configured to receive and discharge air can be efficiently implemented, and thus cooling or heating performance is improved, with the result that a range between cooling temperature and heating temperature is further increased and the times required for cooling and heating can be reduced.

One fan 39 or a plurality of fans 39 may be mounted in the cover plate 40 located on the top surface of the thermoelectric unit 36. The number of fans 39 mounted in the cover plate 40 located on the top surface of the thermoelectric unit 36 may be appropriately selected within a range satisfying the ratio between the sectional areas of the cover plate 40 and the thermoelectric unit 36. More specifically, the cover plate 40 located on the top surface of the thermoelectric unit 36 may include one to four fans, for example, one to three fans, or two or three fans, but the number of fans is not limited thereto.

Furthermore, the cover plate 40 may be located not only on the top surface of the thermoelectric unit 36 but also on at least one of the front surface, back surface, left side surface and right side surface of the thermoelectric unit 36. The cover plate 40 present at the location may include at least one fan 39, at least one hole, or a combination thereof. Although an embodiment in which the cover plate 40 is located the top surface and the two side surfaces of the thermoelectric unit 36 is shown in FIGS. 2 and 3, this merely illustrates one form, but the present invention is not limited thereto.

More specifically, the cover plate 40 may be located on all the top surface, left side surface and right side surface of the thermoelectric unit 36. In this case, the fan 39 may be mounted in the cover plate 40 located on the top surface of the thermoelectric unit 36, and the fan 39 and the hole 41 may be formed in the cover plate 40 located on the left side surface and right side surface of the thermoelectric unit 36, respectively. The cover plate 40 is located on all the top surface and the two side surfaces and seals some surfaces of the thermoelectric unit 36, and thus a passage system configured to receive and discharge air can be efficiently formed, thereby improving the cooling or heating performance of the thermoelectric unit 36. In other words, the cover plate 40 is further located not only on the top surface of the thermoelectric unit 36 but also on the two side surfaces thereof, and thus the entry of air can be induced in a direction from a lower location rather than in directions from the side surfaces, with the result that the discharge of air can be more rapidly performed via the fans 39 located in the top surface of the thermoelectric unit 36. Accordingly, a wider temperature range within which cooling or heating can be performed can be secured and cooling or heating can be rapidly performed by the system in which the above air passage is secured, and thus the present invention can be usefully applied to medical equipment requiring the maintenance of body temperature and a function, such as therapeutic hypothermia.

According to one embodiment, the total number of fans 39 mounted in the cover plate 40 located on the faceable surfaces of the thermoelectric unit 36 may range from 1 to 8, inclusive of one or more fans 39 mounted in the cover plate 40 located on the top surface of the thermoelectric unit 36, for example, from 2 to 8, from 3 to 7, from 4 to 7, from 4 to 6, or from 4 to 5, but is not limited thereto. In other words, the cover plate 40 may be located on the top surface and the two side surfaces of the thermoelectric unit 36. In this case, one or two fans 39 may be mounted only in the top surface, one or more fans 39 may be mounted in each of the top surface and one side surface, or one or more fans 39 may be mounted in each of the top surface and the two side surfaces. When the total number of fans 39 falls within the range, the entry and discharge of air are more rapidly and efficiently performed, and thus cooling or heating performance is considerably improved. Accordingly, a temperature range from a minimum temperature to which cooling can be performed to a maximum temperature to which heating can be performed can be increased, and rapid cooling or heating can be performed.

The operation of the fans 39 may be controlled by the control unit 60.

The cover plate 40 may be located at a distance from the faceable faces of the thermoelectric unit 36 including the top surface of the thermoelectric unit 36. When the cover plate 40 is spaced apart from the thermoelectric unit 36 by a predetermined distance, an entry passage configured to receive external air and a discharge passage configured to discharge entering air are further secured, and thus the air passage system can be more efficiently implemented. Accordingly, the cooling or heating performance of the thermoelectric unit 36 can be considerably improved.

Furthermore, the parts of the cover plate 40 in which the fans 39 are not mounted may have holes 41. In this case, the total number of holes 41 may range from one to two or more, for example, from 1 to 30, from 2 to 20, from 5 to 15, or from 10 to 15, but is not limited thereto. The air passages via which external air is received through the cover plate 40 having a number of holes 41 falling within the number of range and air is discharged through the fans 39 located on the top surface of the thermoelectric unit 36, and thus the performance of the thermoelectric element can be improved.

According to one embodiment, the cover plate 40 including the holes 41 may further include roofs (not shown) above the holes 41. Although the roofs may be formed in, for example, a shade shape, the roofs are not limited to a particular shape as long as they have a roof structure configured to block air entering in a direction from a location above the cover plate 40 and are installed above the holes 41. When the cover plate 40 is located on at least one of the front surface, back surface, left side surface and right side surface of the thermoelectric unit 36, for example, at least one of the left side surface and right side surface of the thermoelectric unit 36, has the holes 41 with roofs, air can be guided through entry in a direction from a lower location, and thus the discharge of air can be more rapidly performed via the fan 39 located in the top surface of the thermoelectric unit 36. Accordingly, the cooling or heating performance of the thermoelectric unit can be further improved.

A method of supporting the cover plate 40 on a support surface is not limited to a particular one. For example, the cover plate 40 may be supported by the support plate 43 located on a support surface. In this case, the cover plate 40 may be coupled to the support plate 43 by means of a screw. The shape of the support plate 43 is not limited to a particular form as long as the support plate 43 can support the cover plate 40 and provides an open space so that the cartridge part 32 below the cover plate 40 can be selectively attached and detached. Furthermore, the cover plate 40 may be formed in a shape in which posts configured to support the cover plate 40 on a support surface are attached to the edges of the shape.

The cartridge part 32 located below the above-described body part 34 is a part through which water supplied from the chamber unit 20 flows. Water is cooled or heated to a set temperature via the thermoelectric unit 36 including the thermoelectric element 37 while passing through the cartridge part 32.

More specifically, the cartridge part 32 may include a flow passage body 33 configured such that a passage configured such that water flows therethrough, i.e., a flow passage, is formed therein, and a temperature sensor 35 located on the bottom surface of the flow passage body 33.

More specifically, the flow passage body 33 may include an inlet 44 configured to receive water entering from the chamber unit 20, a flow passage region (not shown) configured such that a flow passage 31 configured to cool or heat water is formed therein, and an outlet 46 configured to discharge the cooled or heated water.

The chamber unit 20 and the flow passage body 33 of the cartridge part 32 are connected to each other via a water circulation line 45 in order to cool or heat and circulate the supplied water, and the water circulation line 45 may connect the flow passage body 33 of the cartridge part 32 and the pad unit 50 to each other and may also connect the pad unit 50 and the chamber unit 20 to each other.

Check valves 47 may be formed in the inlet 44 and outlet 46 of the flow passage body 33, respectively. When the flow passage body 33 of the cartridge part 32 and the water circulation line 45 are separated from each other, water can be prevented from flowing by the check valves 47. Since the details of the check valves are apparent to those skilled in the art, a description thereof is omitted here.

Although the flow passage 37 formed in the flow passage region may have a zigzag structure, the structure of the flow passage 37 is not limited thereto.

More specifically, the temperature sensor 35 may include a first temperature sensor S1 located in a space from the bottom surface of the flow passage body 33 to the outlet of the flow passage body 33, and a second temperature sensor S2 located in a space from the bottom surface of the flow passage body 33 to the inlet of the flow passage body 33. The first temperature sensor S1 is a component configured to measure the temperature of the cooled or heated water, and the temperature of water entering into the pad unit 50 can be measured via the first temperature sensor S1. Furthermore, the second temperature sensor S2 is a component configured to measure the temperature of water entering back into the temperature control unit 30 after circulating through the pad unit 50, and the temperature of water exiting after circulating through the pad unit 50 can be measured via the second temperature sensor S2. As described above, the two temperature sensors are mounted at different locations below the bottom surface of the flow passage body 33, so that the temperature of water entering into the pad unit 50 and the temperature of water exiting after circulating through the pad unit 50 can be measured, and so that the average of the two temperatures can be automatically calculated and the difference between a temperature set by a user and the temperature of water entering into the pad unit 50 can be minimized, thereby enabling the body temperature of a patient to be efficiently maintained. In other words, in order to calculate the temperature reached through the loss of heat during the circulation through the pad unit 50 and maximally accurately transfer a set temperature value to a patient, design can be made such that the average value of the temperature on the inlet side of the flow passage body 33 and the temperature on the outlet side of the flow passage body 33 can be adjusted to the set temperature.

The first temperature sensor S1 and the second temperature sensor S2 may be controlled via the control unit 60.

The ratio between sectional areas based on the facing surfaces of the flow passage body 33 and the thermoelectric unit 36 may range from 1:1 to 1:1.5, for example, from 1:1 to 1:1.4, from 1:1 to 1:1.3, from 1:1 to 1:1.2, or from 1:1 to 1:1.1, but is not limited thereto. When the ratio between the sizes of the flow passage body 33 and the thermoelectric unit 36 falls within the range, the cooling or heating of water can be rapidly performed, the temperature to which cooling can be performed can be further decreased, and the temperature to which heating can be performed can be further increased.

In the apparatus for controlling body temperature according to one embodiment, when measurements are made by the temperature control unit 30, the time taken to cool water from 25° C. to 8° C. via the above-described temperature control unit 30 may range from 10 to 30 minutes, for example, from 15 to 30 minutes, from 18 to 30 minutes, or from 20 to 28 minutes. As described above, the apparatus for controlling body temperature according to one embodiment can considerably reduce cooling or heating time, and thus the maintenance of normal body temperature and the control of body temperature for therapeutic hypothermia or the like can be rapidly and accurately performed, with the result that the present invention can be usefully used in medical fields.

Referring back to FIG. 1, the circulation unit (not shown) may include the water circulation line 45 so that water can be circulated such that cooled or heated water discharged from the temperature control unit 30, more specifically the flow passage body 33 of the cartridge part 32, passes through the pad unit 50 and flows back into the temperature control unit 30.

In other words, the water circulation line 45 may include all of a passage through which water discharged from the outlet of the chamber unit 20 flows into the temperature control unit 30, more specifically the inlet of the flow passage body 33 in the cartridge part 32, a passage through which water discharged from the outlet of the flow passage body 33 flows into the pad unit 50, a passage through which water is circulated through the pad unit 50, and a passage through which water exiting from the pad unit 50 after circulation flows into the inlet of the chamber unit 20, more specifically the second inlet.

The circulation unit may further include a port (not shown) located outside the pad unit 50 so that the part of the water circulation line 45 contained in the pad unit 50 and the part not contained in the pad unit 50, i.e., the parts of the water circulation lines 45 connected to the chamber unit 20 and the temperature control unit 30, can be separated from and connected to each other.

The water circulation line 45 may be connected to the second inlet and outlet of the chamber unit 20, and may be also connected to the inlet and outlet of the temperature control unit 30. Furthermore, the water circulation lines 45 contained in the pad unit 50 and the water circulation line 45 disposed outside the pad units 50 may be connected to each other via the port (not shown). More specifically, the two water circulation lines 45 contained in the pad unit 50 and configured to have the direction in which water flows into the pad unit 50 and the direction in which water is discharged from the pad unit 50 and the two water circulation lines 45 disposed outside the pad unit 50 and configured to have the direction in which water flows into the pad unit 50 and the direction in which water is discharged from the pad unit 50 may be connected to each other via the port. All the connected portions of the circulation lines 45 may be formed in a detachable form. Since all the connected portions of the circulation lines 45 may be formed in a detachable form, replacement can be easily performed, and thus tab water can be used without the risk of the erroneous operation or stoppage of the operation of the apparatus attributable to sediments or the like, thereby increasing the convenience of a user. The shape of the connected portions of the circulation lines circulation unit is not particularly limited.

Furthermore, a single check valve (not shown) may be formed in the port. When the water circulation lines 45 contained in the pad unit 50 and the water circulation lines 45 disposed outside the pad unit 50 are separated from each other, water can be prevented from flowing out by the check valve.

The operation of the circulation unit may be controlled by the control unit 60.

The pad unit 50 may contain part of the water circulation lines 45. For example, part of the water circulation lines 45 may be contained in the pad unit 50, and may be integrated with the pad unit 50.

The cooled or heated water discharged from the temperature control unit 30, more specifically the flow passage body 33 of the cartridge part 32, flows into and is circulated through the pad unit 50, thereby increasing or decreasing the body temperature of a human who come into contact with the pad unit 50.

The pad unit 50 may be modified into various sizes and shapes according to the characteristics of various body regions requiring the control of body temperature. Furthermore, a plurality of pad units 50 may be used for simultaneous use for various body regions. In this case, a plurality of pad units 50 in which the water circulation lines 45 are contained may be connected to a plurality of corresponding ports.

The water circulation lines 45 contained in the pad unit 50 may be formed in a zigzag structure, a honeycomb structure, or a combination structure thereof. Furthermore, when the water circulation lines are formed in a zigzag structure, they may be formed in a structure that is parallel to the direction in which water is injected through the inlet of the pad unit 50, may be formed in a structure that is vertical to the direction in which water is injected, or may be formed in the combination structure thereof. The structure of the water circulation line 45 contained in the pad unit 50 is not limited thereto. According to one embodiment, the pad unit 50 through which the cooled or heated water is circulated may have an adjusted temperature ranging from 4 to 50° C. In other words, when the temperature of the pad unit 50 through which cooled water is circulated is measured, a minimum temperature of 4° C. may be obtained. Furthermore, when the temperature of the pad unit 50 through which heated water is circulated is measured, a maximum temperature of 50° C. may be obtained. The pad unit 50 through which cooled or heated water is circulated may have an adjusted temperature ranging, for example, from 5 to 50° C., from 5 to 49° C., from 6 to 49° C., from 6 to 48° C., or from 7 to 48° C. Furthermore, the difference between a maximum temperature and a minimum temperature within the adjusted temperature range of the pad unit 50 may range from 41 to 46° C., for example, from 42 to 46° C., from 43 to 46° C., from 44 to 46° C., or from 45 to 46° C. As described above, the apparatus for controlling body temperature according to one embodiment can secure a wider temperature adjustment range from a minimum temperature to which cooling can be performed and a maximum temperature to which heating can be performed, i.e., the larger difference between a minimum temperature to which cooling can be performed and a maximum temperature to which heating can be performed, and thus the maintenance of normal body temperature and the control of body temperature for therapeutic hypothermia or the like can be rapidly and accurately performed, thus being usefully used in medical fields.

The control unit 60 may control the operation of the temperature control unit 30. In other words, the control unit 60 may control the operations of the first temperature sensor S1 mounted in the cartridge part 32 of the temperature control unit 30, more specifically located on the outlet side of the flow passage body 33 below the bottom surface of the flow passage body 33, and the second temperature sensor S2 located on the inlet side of the flow passage body 33.

The control unit 60 may control the operations of the first temperature sensor S1 and the second temperature sensor S2, and may also control various operations applied to the apparatus for controlling body temperature. For example, a separate probe configured to directly measure the body temperature of a patient may be added to the apparatus for controlling body temperature. The control unit 60 may control the operation of the probe, may control the operation of automatically performing cooling or heating in accordance with the body temperature of a patient, may control the operation of a timer configured to set cooling or heating time, and may control the calculation of a converted value based on the unit of temperature. However, the control of the control unit 60 is not limited thereto.

Since the details of the design of the control unit are apparent to those skilled in the art, a description thereof is omitted.

### MODE FOR INVENTION

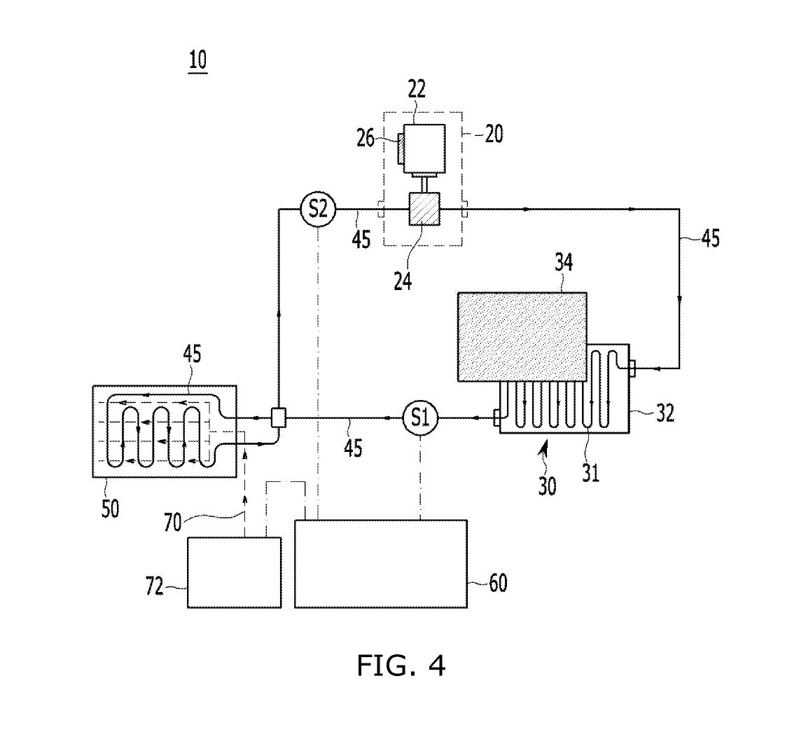

An apparatus for controlling body temperature according to one embodiment may further include an air unit in order to prevent bedsores. The apparatus for controlling body temperature, to which the air unit has been added, will be described with reference to FIG. 4. The drawing to which reference will be made merely corresponds to another form of the apparatus for controlling body temperature according to one embodiment, and the present invention is not limited thereto.

FIG. 4 is a schematic diagram showing the configuration of an apparatus for controlling body temperature using a thermoelectric element according to another embodiment. Descriptions of the reference symbols of FIG. 4 that are the same as the reference symbols of FIG. 1 will be omitted.

Referring to FIG. 4, the apparatus 10 for controlling body temperature according to one embodiment may further include an air unit (not shown). The air unit may include an airline 70 that corresponds to a passage for air. The airline 70 is formed at a location independent of the location of the water circulation line 45s, and thus the movement of air and the circulation of water can be performed independently without interference with each other. Via the airline 70, air may be injected in one direction, and air may be discharged in the opposite direction.

Accordingly, according to one embodiment, the process of injecting and discharging air into and from the pad unit 50 for a predetermined time is repeated via the airline 70 of the air unit, and thus bedsores of a patient may be maximally prevented. Since the material of a conventional mat capable of preventing bedsores is chiefly nonwoven fabric, air is naturally discharged after being injected. In this case, the body region of a patient that comes into contact with the mat while air is being injected remains the same while air is being discharged, and thus there is a limitation on the prevention of a bedsore. According to one embodiment, a cushioning sensation is continuously adjusted by repeating the injection and discharge of air for a predetermined time, and thus a contact point between a mat and the body region of a patient varies, thereby considerably improving bedsore prevention performance.

Part of the airline 70 may be contained in the pad unit 50 independently of the water circulation line 45. For example, the airline 70 and the water circulation line 45 may be disposed in a structure in which they are disposed in different layers within the pad unit 50, as shown in FIG. 4, or may be disposed in independent structures in the same layer. Furthermore, although the structure of the airline 70 contained in the pad unit 50 may be a fork structure parallel to the direction in which air is injected, as shown in FIG. 4, it is not limited thereto.

The air unit may further include an air manipulation unit 72 configured to enable the injection and discharge of air to be repeated. More specifically, when the air manipulation unit 72 is operated, for example, by a method of pressing the button of the air manipulation unit 72, air may be injected and then a valve may be automatically opened for a set time to thus discharge air, or air may be forcibly injected and then discharged, and this process may be repeatedly performed.

The operation of the air unit, more specifically the air manipulation unit 72, may be controlled by the control unit 60.

The apparatus for controlling body temperature according to one embodiment may be fabricated as a small-sized portable type for use in an ambulance, an ambulance bed, etc., or may be fabricated as a stand-type for use in the operating room, intensive care unit, recovery room, etc. of a hospital. Furthermore, an embedded battery may be mounted in the portable type or stand-type apparatus in order to prepare for a case where power cannot be supplied or the supply of power is stopped.

Although the preferred embodiments of the present invention have been described above, the present invention is not limited thereto. The present invention may be modified into various forms and then practiced within the range of the claims, the detailed description of the invention, and the accompanying drawings, and it will be apparent that the various forms also fall within the scope of the present invention.

1. An apparatus for controlling body temperature using a thermoelectric element, the apparatus comprising:
a chamber unit comprising a water container configured to store water;
a temperature control unit configured to cool or heat water supplied from the chamber unit;
a circulation unit comprising a water circulation line configured such that the cooled or heated water discharged from the temperature control unit is circulated therethrough to flow back to the temperature control unit;
a pad unit configured to contain a part of the water circulation line and to increase or decrease body temperature of a human who comes into contact therewith; and
a control unit configured to control an operation of the temperature control unit;
wherein the temperature control unit comprises a cartridge part and a body part located on a top surface of the cartridge part, and the body part comprises a thermoelectric unit comprising a thermoelectric element located on the top surface of the cartridge part and a heat sink plate located on a top surface of the thermoelectric element; and
wherein the pad unit through which the cooled or heated water is circulated has an adjusted temperature ranging from 4 to 50° C., and a difference between a maximum temperature and a minimum temperature within an adjusted temperature range of the pad unit ranges from 41 to 46° C.

2. The apparatus of claim 1, wherein a time required to cool water from 25° C. to 8° C. via the temperature control unit ranges from 10 to 30 minutes.

3. The apparatus of claim 1, wherein the chamber unit further comprises a sensor located independently of the water container, an indicator mounted on a side surface of the water container, or a combination thereof.

4. The apparatus of claim 1, wherein:
the body part further comprises a cover plate located on at least one of faceable surfaces of the thermoelectric unit; and
the cover plate is located on at least a top surface of the thermoelectric unit, and the cover plate located on the top surface of the thermoelectric unit comprises a fan.

5. The apparatus of claim 4, wherein a ratio between sectional areas based on facing surfaces of the cover plate located on the top surface of the thermoelectric unit and the thermoelectric unit ranges from 1:1 to 1.5:1.

6. The apparatus of claim 4, wherein the cover plate located on the top surface of the thermoelectric unit comprises one to four fans.

7. The apparatus of claim 4, wherein the cover plate is located on the top surface, a left side surface, and a right side surface of the thermoelectric unit.

8. The apparatus of claim 4, wherein the cover plate is located at a distance from the faceable surfaces comprising the top surface of the thermoelectric unit.

9. The apparatus of claim 4, wherein the cover plate further located on at least one of the front surface, a back surface, a left side surface, and a right side surface of the thermoelectric unit comprises at least one fan, at least one hole, or a combination thereof.

10. The apparatus of claim 9, wherein the cover plate comprising the hole further comprises a roof above the hole.

11. The apparatus of claim 1, wherein the cartridge part has a structure that is detached from the body part.

12. The apparatus of claim 1, wherein the cartridge part comprises:
a flow passage body comprising an inlet configured to receive water, a flow passage region configured such that a flow passage configured to cool or heat water is formed, and an outlet configured to discharge the cooled or heated water; and
temperature sensors located below a bottom surface of the flow passage body.

13. The apparatus of claim 12, wherein the temperature sensors comprise:
a first temperature sensor located on an outlet side below the bottom surface of the flow passage body, and configured to measure a temperature of the cooled or heated water; and
a second temperature sensor located on an inlet side below the bottom surface of the flow passage body, and configured to measure a temperature of water that flows into the temperature control unit after being circulated through the pad unit.

14. The apparatus of claim 12, wherein a ratio between sectional areas based on facing surfaces of the flow passage body and the thermoelectric unit ranges from 1:1 to 1:1.5.

15. The apparatus of claim 1, wherein the part of the water circulation line contained in the pad unit has a zigzag structure, a honeycomb structure, or a combination structure thereof.

16. The apparatus of claim 1, wherein:
the apparatus for controlling body temperature further comprises an air unit comprising an airline;
the pad unit further contains a part of the airline independently of the water circulation line; and
the control unit further controls an operation of the air unit.

17. The apparatus of claim 16, wherein the air unit further comprises an air manipulation unit configured to enable entry and discharge of air to be repeatedly performed.

18. The apparatus of claim 16, wherein the part of the airline contained in the pad unit has a fork structure in a direction parallel to a direction in which air is injected.
